(19) United States
(12) Patent Application Publication
(10) Pub. No.: US 2014/0058450 A1
(43) Pub. Date: Feb. 27, 2014

(54) SPINAL CORRECTION SYSTEM AND METHOD

(71) Applicant: WARSAW ORTHOPEDIC, INC., Warsaw, IN (US)

(72) Inventor: Vincent Arlet, Philadelphia, PA (US)

(21) Appl. No.: 13/912,967

(22) Filed: Jun. 7, 2013

Related U.S. Application Data
(60) Provisional application No. 61/692,119, filed on Aug. 22, 2012.

(51) Int. Cl. A61B 17/70

(52) U.S. Cl. CPC A61B 17/70
USPC 606/256

(57) ABSTRACT

A spinal construct comprises a first member defining a longitudinal axis and extending between a first end and a second end being offset relative to the longitudinal axis. A second member defines a longitudinal axis and extends between a first end and a second end being offset relative to the longitudinal axis of the second member. The second ends are connected to define an axis of rotation such that the members are relatively rotatable about the second ends and the axis of rotation is offset relative to the longitudinal axes. Systems and methods are disclosed.

### CROSS-REFERENCE TO RELATED APPLICATION

This application claims the benefits of U.S. Provisional Patent Application No. 61/692,119 filed Aug. 22, 2012, the contents of which being hereby incorporated in its entirety by reference.

### TECHNICAL FIELD

The present disclosure generally relates to medical devices for the treatment of musculoskeletal disorders, and more particularly to a surgical system and method for correction of a spine disorder.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as, for example, spinal constructs and interbody devices, for stabilization of a treated section of a spine. This disclosure describes an improvement over these prior art technologies.

### SUMMARY

In one embodiment, a spinal construct is provided. The spinal construct comprises a first member defining a longitudinal axis and extending between a first end and a second end being offset relative to the longitudinal axis. The first member is configured for attachment to at least a first vertebra of a body. A second member is configured for attachment to at least a second vertebra of the body spaced from the first vertebra and defining a longitudinal axis. The second member extends between a first end and a second end being offset relative to the longitudinal axis of the second member. The second ends are connected to define an axis of rotation such that the members are relatively rotatable about the second ends and the axis of rotation is offset relative to the longitudinal axes. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a surgical system in accordance with the principles of the present disclosure;

FIG. 2 is a side view, in part cross section, of the components shown in FIG. 1;

FIG. 3 is a side view of the components shown in FIG. 1;

FIG. 4 is a side view of the components shown in FIG. 1;

FIG. 5 is a side view of components of one embodiment of a surgical system in accordance with the principles of the present disclosure disposed with vertebrae;

FIG. 6 is a side view of the components and vertebrae shown in FIG. 5;

FIG. 7 is a side view of components of one embodiment of a surgical system in accordance with the principles of the present disclosure disposed with vertebrae;

FIG. 8 is a side view of components of one embodiment of a surgical system in accordance with the principles of the present disclosure disposed with vertebrae;

FIG. 9 is a side view of components of one embodiment of a surgical system in accordance with the principles of the present disclosure disposed with vertebrae; and

FIG. 10 is a plan view of components of one embodiment of a surgical system in accordance with the principles of the present disclosure disposed with vertebrae.

### DETAILED DESCRIPTION

The exemplary embodiments of the system and related methods of use disclosed are discussed in terms of medical devices for the treatment of musculoskeletal disorders and more particularly, in terms of a surgical system and method for correction of a spine disorder. In one embodiment, the surgical system comprises a spinal construct, such as, for example, a temporary rod for a posterior vertebral column resection. In some embodiments, the surgical system can be employed with a posterior vertebral column resection to correct angular and fixed kyphotic deformity, such as post traumatic deformity, congenital deformity and/or post infectious deformity.

In some embodiments, the surgical system can be employed with a posterior vertebral column resection from a posterior approach to provide circumferential control of a spinal cord during correction maneuvers. The present surgical system and method resist and/or prevent excessive lengthening of a spinal cord and/or excessive compression of the spinal cord.

In one embodiment, the surgical system comprises a spinal construct having a fulcrum of correction disposed at a level of the spinal cord to resist and/or prevent excessive lengthening of a spinal cord and/or excessive compression of the spinal cord. In one embodiment, the surgical system comprises a spinal construct comprising a temporary rod or an implantable rod that includes two rod portions linked with a ratcheted fulcrum.

In one embodiment, the surgical system comprises a spinal construct having an axis of rotation at the level of an anterior portion of a spinal cord. In one embodiment, the surgical system comprises a spinal construct and pedicle screws implanted in the posterior lamina as deep as possible so that the center of rotation of the spinal construct is as close as possible to the anterior portion of the spinal cord. In some embodiments, the spinal construct is curved close to a hinged ratchet mechanism of the spinal construct to dispose the center of rotation as close as possible to the level of the anterior portion of the spinal cord. For example, as the spinal construct is inserted after, for example, laminectomy and pediculectomy, the hinged ratchet mechanism is disposed parallel to the spine and close to the anterior aspect of the cord.

In one embodiment, the spinal construct includes visual indicia, which may include etching, to display distance for identifying shortening and/or lengthening of the spine during correction. In one embodiment, the spinal construct includes a locking mechanism with gear teeth to resist and/or prevent loss of correction at the level of a knob connected with the locking mechanism. In one embodiment, the surgical system comprises a rod bender that can bend the spinal construct in a coronal plane. In one embodiment, the spinal construct includes a two plate part of the ratchet mechanism to avoid any impingement on the spinal cord. In one embodiment, the spinal construct includes a medial rod that does not protrude medially towards the spinal canal more than a crown of a pedicle screw that protrudes medially. As such, a lateral rod of the spinal construct has a coronal offset at the level of the plate that is greater than the medial rod. In one embodiment, the locking mechanism is disposed between two rods and includes gear teeth that lock the two plates. In one embodiment, the teeth allow a gradual and/or incremental rotation from 5 degrees for each tooth to perform gradual correction.

In some embodiments, the surgical system includes a cantilever for engagement with the hinged ratchet mechanism of the spinal construct to reduce kyphosis between the rod portions. In one embodiment, the spinal construct includes a spring loaded mechanism that disposes the gear teeth between an unlocked and non-engaged orientation and a locked and engaged orientation to tighten relative orientation of the spinal construct. In some embodiments, the spinal construct is fabricated from chrome cobalt and titanium. In some embodiments, the spinal construct has a diameter such as 5.5 millimeters (mm), 6 mm or 6.3 mm. In some embodiments, the spinal construct comprises a provisional spinal construct and/or a working spinal construct that can be left in place with vertebrae or switched to permanently implantable spinal constructs.

In one embodiment, the spinal construct includes hinged rod members having a center of rotation of the members that is anterior to an axis of the members. In one embodiment, the center of rotation of the spinal construct closely approximates the center of rotation of vertebral bodies. This configuration resists and/or prevents excessive stretching or compression in the spinal cord when deformity is corrected.

In some embodiments, the surgical system includes instruments and tools for correcting a sagittal deformity and rebalancing a spine of a body. In one embodiment, the surgical system is employed to treat degenerative deformities of a spine in a sagittal plane. In some embodiments, the surgical system is employed to treat disorders that create an unbalance of a body and loss of alignment between body parts. In one embodiment, the surgical system provides a selected amount of correction to apply a selected balance to a spine and provides control and adjustment to the amount of correction. In one embodiment, the surgical system includes a series of tools and instruments that allow formulation of a type of correction applied and can control the correction stabilization using posterior instrumentation.

In one embodiment, the surgical system facilitates permanently implanting spinal constructs according to the contour of the spine in a sagittal plane of a body. In one embodiment, the surgical system evaluates an angle between vertebra to contour a template spinal construct. For example, the surgical system and method can include a temporary holding spinal construct system with an angular adjustment.

In one embodiment, the surgical system and method include an apparatus for stabilizing a musculoskeletal structure that includes a spinal construct having medial rod pivotally connected to a lateral rod such that angular adjustment can be made and held therebetween. The angular adjustment can be locked in place by a locking mechanism, which permits the angular adjustment to be selectively fixed or held in an orientation. The rods may be connected to portions of the musculoskeletal structure, and the angular adjustment of the rods is made to accommodate an existing or configured angular difference between portions of the musculoskeletal structure. In one embodiment, the surgical system includes a temporary spinal construct that has the ability to lock and maintain an angle between both ends for employment with procedures, such as, for example, pedicle subtraction osteotomy (PSO) and vertebral column resection (VCR).

In one embodiment, multiple spinal constructs are employed concurrently along a same section of a spine in, for example, a side by side orientation. Once the angular position is determined and set, at least one of the spinal constructs can be removed and employed as a template to bend an implantable spinal construct or a permanent spinal construct. In one embodiment, the spinal constructs may also be implantable.

In one embodiment, the system comprises a spinal implant including an interbody fusion device. In one embodiment, the system comprises a spinal implant including an expandable cage.

In one embodiment, one or all of the components of the surgical system are disposable, peel-pack, pre-packed sterile devices. One or all of the components of the surgical system may be reusable. The surgical system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed surgical system and methods may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

In some embodiments, the system of the present disclosure comprises a spinal implant that includes bone graft, for example, osteograft. As used in the specification and including the appended claims of the present disclosure, the term “bone graft” includes materials that may include both synthetic and natural bone. In some embodiments, natural bone may be taken from the graft recipient, for example, autograft, or may be taken from another source, for example, allograft, such as a cadaver, or can be xenograft, for example, bovine.

In some embodiments, osteoconduction and osteoinduction both contribute to bone formation. Osteoconductivity provides a structural framework or microscopic and macroscopic scaffolding for cells and cellular materials that are involved in bone formation, for example, osteoclasts, osteoblasts, vasculature and mesenchymal cells. Osteoinductive material stimulates differentiation of host mesenchymal cells into chondroblasts and osteoblasts. Natural bone allograft materials can comprise either cortical or cancellous bone. In some embodiments, allografts can comprise mammalian cadaver bone treated to remove all soft tissue, including marrow and blood, and then textured to form a multiplicity of holes of selected size, spacing, and depth. In some embodiments, the textured bone section can be immersed and demineralized, for example, in a dilute acid bath.

In some embodiments, allografts are formed of organic bone matrix with perforations that extend from one surface, through the matrix, to the other surface to provide continuous channels between opposite surfaces. In some embodiments, partially-demineralized cortical bone constructs may be surface-demineralized to prepare the graft to be soaked in bone growth-promoting substances such as bone morphogenetic protein.

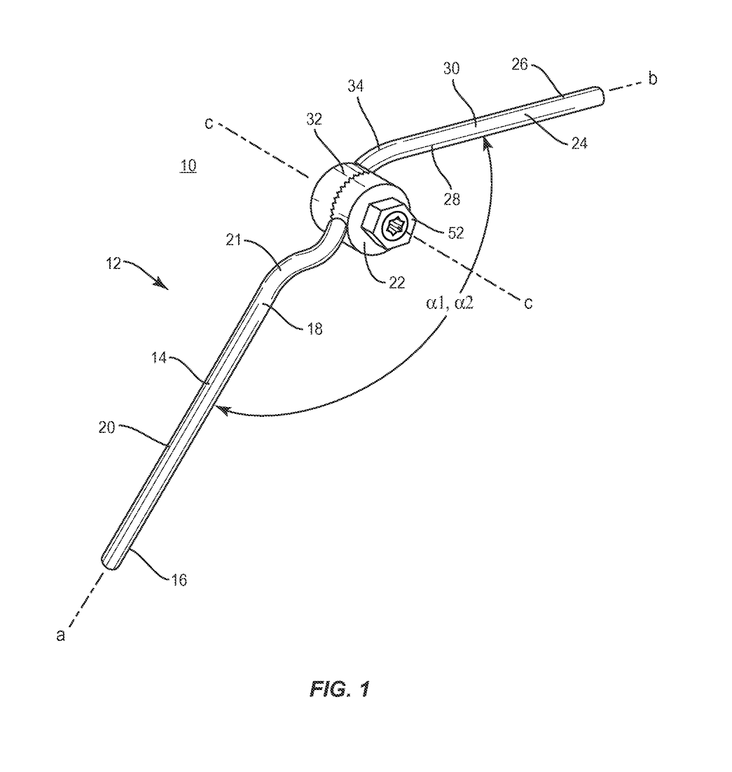

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning to FIGS. 1-4, there are illustrated components of a surgical system, such as, for example, a spinal correction system 10, in accordance with the principles of the present disclosure.

The components of spinal correction system 10 can be fabricated from biologically acceptable materials suitable for medical applications, including metals, synthetic polymers, ceramics and bone material and/or their composites, depending on the particular application and/or preference of a medical practitioner. For example, the components of spinal correction system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of spinal correction system 10 may have material composites, including the above materials, to achieve various desired characteristics such as strength, rigidity, elasticity, compliance, biomechanical performance, durability and radiolucency or imaging preference. The components of spinal correction system 10, individually or collectively, may also be fabricated from a heterogeneous material such as a combination of two or more of the above-described materials. The components of spinal correction system 10 may be monolithically formed, integrally connected or include fastening elements and/or instruments, as described herein.

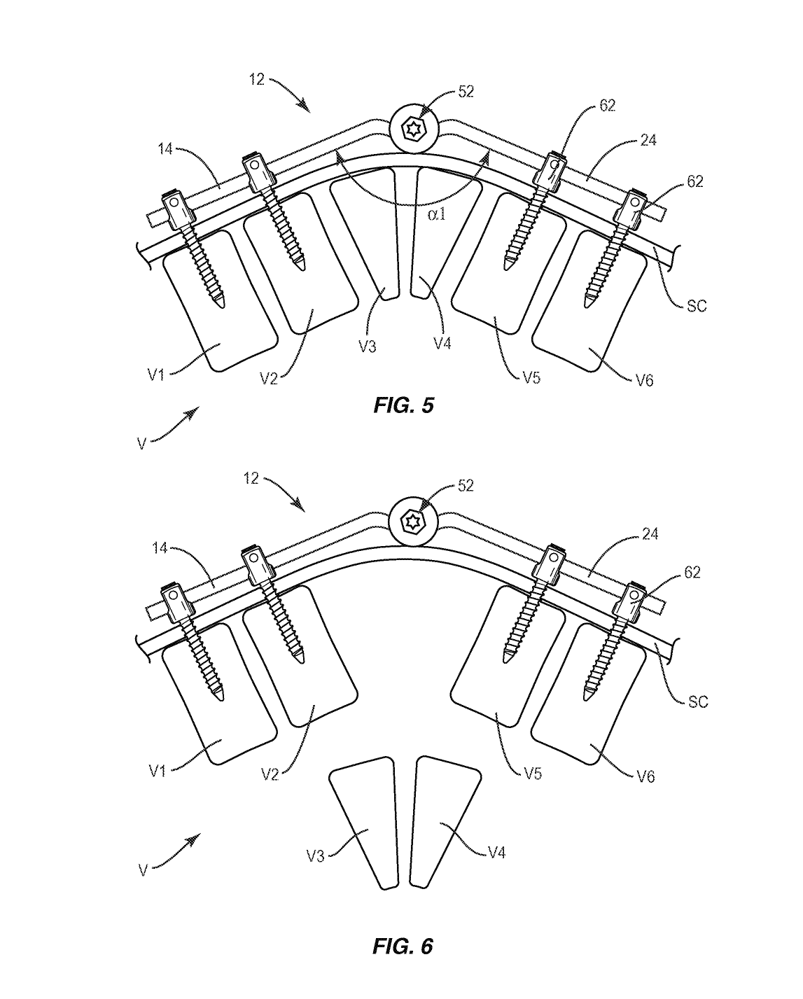

Spinal correction system 10 includes a spinal construct 12 configured for engagement with a spine to correct a spinal disorder, such as, for example, a sagittal deformity, as described herein. Spinal construct 12 is configured for attachment to vertebrae (as shown, for example, in FIGS. 5-10) during surgical treatment of a spinal disorder, examples of which are discussed herein.

Spinal construct 12 includes a member, such as, for example, lateral rod 14 that defines a longitudinal axis a, and extends from an end 16 to an end 18. Rod 14 includes an intermediate portion 20. In some embodiments, end 16 and/or portion 20 are configured for attachment to a posterior portion of vertebrae of a body, as described herein.

End 18 includes a plate, such as, for example, a head 22 having a disc shaped configuration. Portion 20 includes an arcuate portion 21 such that rod 14 includes a curvature adjacent end 18 and head 22 is offset relative to axis a. Head 22 is offset relative to axis a in a plane, such as, for example, a sagittal plane SP of a body, as described herein. Head 22 is also offset relative to axis a in a plane, such as, for example, a coronal plane of a body, as described herein. In some embodiments, head 22 may be disposed at alternate orientations relative to axis a, such as, for example, transverse, perpendicular and/or other angular orientations such as acute or obtuse, co-axial and/or staggered.

Spinal construct 12 includes a member, such as, for example, medial rod 24 that defines a longitudinal axis b, and extends from an end 26 to an end 28. Rod 24 includes an intermediate portion 30. In some embodiments, end 26 and/or portion 30 are configured for attachment to a posterior portion of vertebrae of a body, as described herein.

End 28 includes a plate, such as, for example, a head 32 having a disc shaped configuration. Portion 30 includes an arcuate portion 34 such that rod 24 includes a curvature adjacent end 28 and head 32 is offset relative to axis b. Head 32 is offset relative to axis b in a plane, such as, for example, a sagittal plane SP of a body, as described herein. In some embodiments, head 32 may be disposed at alternate orientations relative to axis b, such as, for example, transverse, perpendicular and/or other angular orientations such as acute or obtuse, co-axial and/or staggered.

In some embodiments, the respective cross-section of rods 14, 24 may have various configurations, for example, round, oval, rectangular, polygonal, irregular, uniform and non-uniform. Rod 14 may have a different cross-sectional area, geometry, material or material property such as strength, modulus or flexibility relative to rod 24. In some embodiments, the respective cross-section of heads 22, 32 may have various configurations, for example, round, oval, rectangular, polygonal, irregular, uniform and non-uniform. Head 22 may have a different cross-sectional area, geometry, material or material property such as strength, modulus or flexibility relative to head 32.

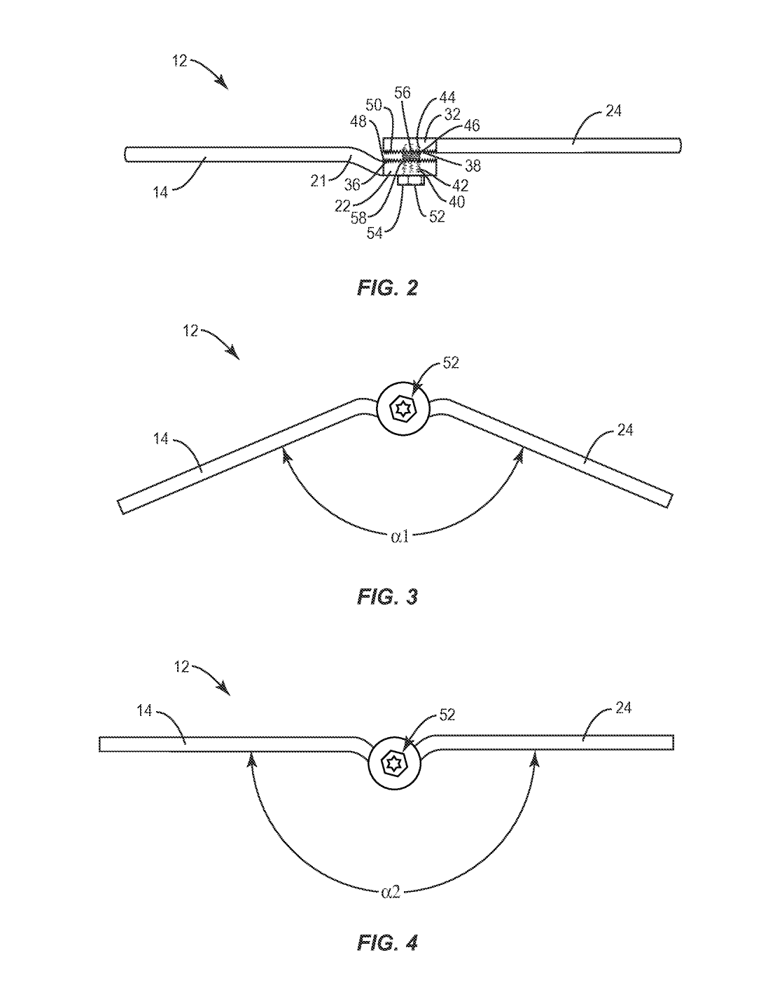

Heads 22, 32 are connected in a hinge configuration with rods 14, 24 and define an axis of rotation c, which is disposed transverse to axes a, b, such that rods 14, 24 are relatively rotatable about heads 22, 32 and axis c is offset relative to axes a, b. Head 22 includes a transverse mating surface 36, as shown in FIG. 2, configured to interface with a transverse mating surface 38 of head 32 to dispose rods 14, 24 in a selected orientation, as described herein. Arcuate portion 21 offsets head 22 laterally to provide spacing for disposal of head 32 upon connection and fixation of heads 22, 32. As such, rods 14, 24 can be rotated in substantially the same plane. In some embodiments, such lateral offset of head 22 facilitates disposal of rods 14, 24 in substantially co-axial alignment.

Head 22 includes an inner surface 40 that defines a centrally disposed opening 42. Opening 42 is configured for passage therethrough of a lock, described herein. Head 32 includes an inner surface 44 that defines a centrally disposed opening 46. Opening 46 is configured for disposal of the lock and surface 44 is threaded for engagement with the lock to draw heads 22, 32 into fixation for disposal of rods 14, 24 in a selected orientation, as described herein.

Surface 36 has a plurality of gear teeth 48 disposed radially thereabout in a splined configuration. Surface 38 has a plurality of gear teeth 50 disposed radially thereabout in a splined configuration. Teeth 48, 50 and/or surfaces 36, 38 are configured to interface for fixation of rods 14, 24 in a selected orientation. In some embodiments, heads 22, 32 comprise a ratchet that includes teeth 48, 50 and/or surfaces 36, 38 such that the rods 14, 24 are incrementally rotatable to one or a plurality of selected orientations. In some embodiments, the ratchet configuration of heads 22, 32 provide relative rotation of rods 14, 24 such that rods 14, 24 are rotatable in 5 degree increments to one or a plurality of selected orientations.

Spinal construct 12 includes a lock, such as, for example, a screw 52. Screw 52 includes an enlarged head 54 and a threaded shaft 56. Shaft 56 is configured for slidable disposal and passage through opening 42. Shaft 56 is configured for disposal with opening 46 and threadably engageable with surface 44 to connect, attach, fix and/or lock, provisionally, removably and/or permanently, head 22 with head 32. Head 54 engages an outer surface of head 22 such that threaded engagement of shaft 56 with surface 44 draws shaft 56 through openings 42, 46 and teeth 48, 50 and/or surfaces 36, 38 into fixation to dispose rods 14, 24 in one or a plurality of selected orientations and/or to draw the components of spinal construct 12 together and secured as a unit, as shown in FIG. 1. The lock is disposable between a non-locking orientation, as shown in FIG. 2, such that teeth 48, 50 are not fixedly engaged to prevent relative rotation of rods 14, 24 and a locking orientation, as shown in FIG. 1, such teeth 48, 50 are disposed in a fixed engagement and rods 14, 24 are prevented from relatively rotating.

Screw 52 may be threadably removed from heads 22, 32 to disassemble the components of spinal construct 12 to, for example, adjust spinal construct 12 for gradual correction of vertebrae with incremental rotation of rods 14, 24 to one or a plurality of selected orientations. Assembly of the components can be performed prior to delivery to a surgical site and/or in situ, including subsequent to fixation of rods 14, 24 with vertebrae. In some embodiments, the lock of spinal construct 12 includes a resiliently biased member, such as, for example, a coil spring 58 disposed about shaft 56 and within openings 42, 46. Spring 58 engages the surfaces of heads 22, 32 in a configuration to bias the lock to the non-locking orientation. The bias of spring 58 is overcome by threading screw 52 with heads 22, 32, as described herein, to dispose the lock in the locking orientation. In one embodiment, the biasing member includes an axial element, such as, for example, a flexible rod disposed between heads 22, 32. In one embodiment, the biasing member has a solid disc shape disposed between heads 22, 32. In one embodiment, the biasing member has a tubular wall disposed between heads 22, 32. In some embodiments, the biasing member may include an elastomeric member, clip, leaf spring, gravity induced configuration, pneumatic configuration, hydraulic configuration and/or manual lever.

In operation, spinal construct 12 is disposed in a non-locking orientation with screw 52, as described herein, such that rods 14, 24 are relatively rotatable. The components of spinal construct 12 are movable such that rods 14, 24 are relatively rotatable in a plane, such as, for example, a sagittal plane of a body, between a first configuration, for example as shown in FIG. 3, such that rods 14, 24 are disposed at a first relative angular orientation, such as, for example, angle α1, and a second configuration, for example as shown in FIG. 4, such that rods 14, 24 are disposed at a second relative angular orientation, such as, for example, angle α2.

Upon disposal of rods 14, 24 in a selected orientation, such as, for example, at angle α1 or α2, screw 52 can be disposed in the locking orientation, as described herein, to fix rods 14, 24 in the selected orientation at angle α1 or α2. In some embodiments, spinal construct 12 is adjustable for gradual correction of vertebrae with incremental rotation of rods 14, 24 to one or a plurality of selected orientations. As such, screw 52 is disposed in the non-locking orientation and rod 24 is incrementally rotated relative to rod 14. Screw 52 is disposed in the locking orientation such that spinal construct 12 provides incremental correction. In some embodiments, spinal construct 12 can be configured as a temporary or provisional construct and replaced with an alternate permanently implantable spinal construct, such as, spinal rods, plates and/or bone fasteners. In some embodiments, spinal construct 12 can remain implanted with vertebrae in a configuration as a permanently implantable construct.

In some embodiments, the adjustability of spinal construct 12 includes relative rotation of rods 14, 24 through an angular range of 0 to 360 degrees. In some embodiments, heads 22, 32 and/or rods 14, 24 can be rotated clockwise and counter-clockwise. In one embodiment, spinal correction system 10 includes a kit or set that includes spinal construct 12 described above and a plurality of alternate sized and/or configured rods 14, 24.

In assembly, operation and use, spinal correction system 10 is employed during a surgical procedure, such as, for example, a PSO, VCR or other correction treatment to treat, for example, scoliosis and/or kyphosis of a spine. In some embodiments, one or all of the components of spinal correction system 10 can be delivered or implanted as a pre-assembled device or can be assembled in-situ. In some embodiments, one or all of the components of spinal correction system 10 may be completely or partially revised, removed or replaced.

As shown in FIGS. 5-10, spinal correction system 10 can be employed with a surgical correction treatment of an applicable condition or injury of an affected section or sections of a spinal column, such as, for example, vertebrae V, which includes a plurality of vertebra V1-V6, and adjacent areas within a body. In one embodiment, spinal correction system 10 is employed with a posterior VCR from a posterior approach to vertebrae V to provide circumferential control of a spinal cord and dura SC during correction maneuvers. The components of spinal correction system 10 resist and/or prevent excessive lengthening of spinal cord SC and/or excessive compression of spinal cord SC.

In use, to treat vertebrae V, a medical practitioner obtains access to a surgical site including vertebra V1-V6 in any appropriate manner, such as through incision and retraction of tissues. In some embodiments, spinal correction system 10 can be used in any existing surgical method or technique including open surgery, mini-open surgery, minimally invasive surgery and percutaneous surgical implantation, whereby a section of vertebrae V including vertebra V1-V6 are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of spinal correction system 10. A preparation instrument (not shown) can be employed to prepare tissue surfaces of vertebrae V, as well as for aspiration and irrigation of a surgical region.

Pilot holes are made in selected vertebra of vertebrae V for receiving fixation elements, such as, for example, bone fasteners 62. Each bone fastener 62 is inserted or otherwise engaged with a particular vertebra. Bone fasteners 62 each include a receiver defining an implant cavity configured for disposal of rods 14, 24. In one embodiment, bone fasteners 62 are implanted in the posterior lamina as deep as possible so that axis c, described herein, is disposed adjacent an anterior portion of spinal cord SC. In some embodiments, spinal correction system 10 may include one or a plurality of fixation elements.

Spinal construct 12 and rods 14, 24 are employed as provisional and/or working rods to temporarily support vertebrae V during a corrective procedure. In some embodiments, spinal correction system 10 may include one or a plurality of spinal constructs 12. In some embodiments, the plurality of spinal constructs 12 may be disposed in various alternate orientations, such as, for example, side by side, parallel, transverse and/or other angular orientations such as acute or obtuse, co-axial and/or may be offset or staggered. In some embodiments, the plurality of spinal constructs 12 may provide a template configuration for permanently implantable spinal rods, such as, implantable, final, permanent, removable, non-removable, bio-absorbable, resorbable and/or bio-degradable.

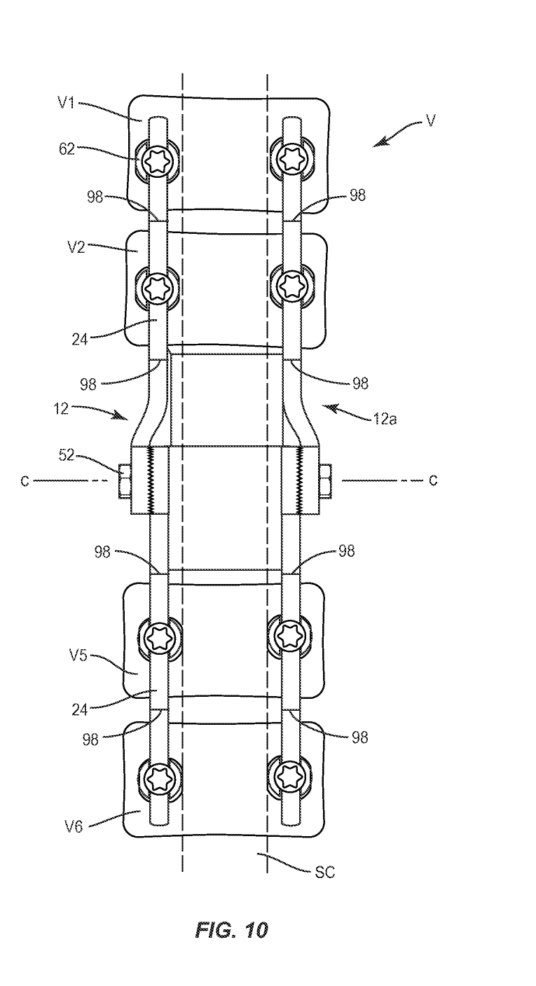

A first spinal construct 12 is attached to vertebrae V, as shown in FIG. 5. Spinal construct 12 is disposed in a non-locking orientation with screw 52, as described herein. A rod 14 of spinal construct 12 is fixed with bone fasteners 62 to vertebrae V1, V2. A rod 24 of spinal construct 12 is rotated relative to rod 14 to an angle α1, measured from their respective axes, a, b, corresponding to the orientation of vertebrae V1, V2 relative to vertebrae V5, V6. A rod 24 of spinal construct 12 is fixed with bone fasteners 62 to vertebrae V5, V6. Spinal construct 12 is disposed in a locking orientation with screw 52, as described herein, to fix rods 14, 24 in the selected orientation at angle α1. A second spinal construct 12a, similar to spinal construct 12, is attached to vertebrae V in a side by side orientation with spinal construct 12, as shown in FIG. 10.

Axis c of spinal constructs 12, 12a are disposed adjacent the anterior portion of spinal cord SC. Arcuate portions 21, 34 facilitate disposal of heads 22, 32 adjacent the anterior portion of spinal cord SC. This configuration resists and/or prevents excessive stretching or compression in spinal cord SC. In one embodiment, as shown in FIG. 6, apical vertebrae V3, V4 are removed from vertebrae V.

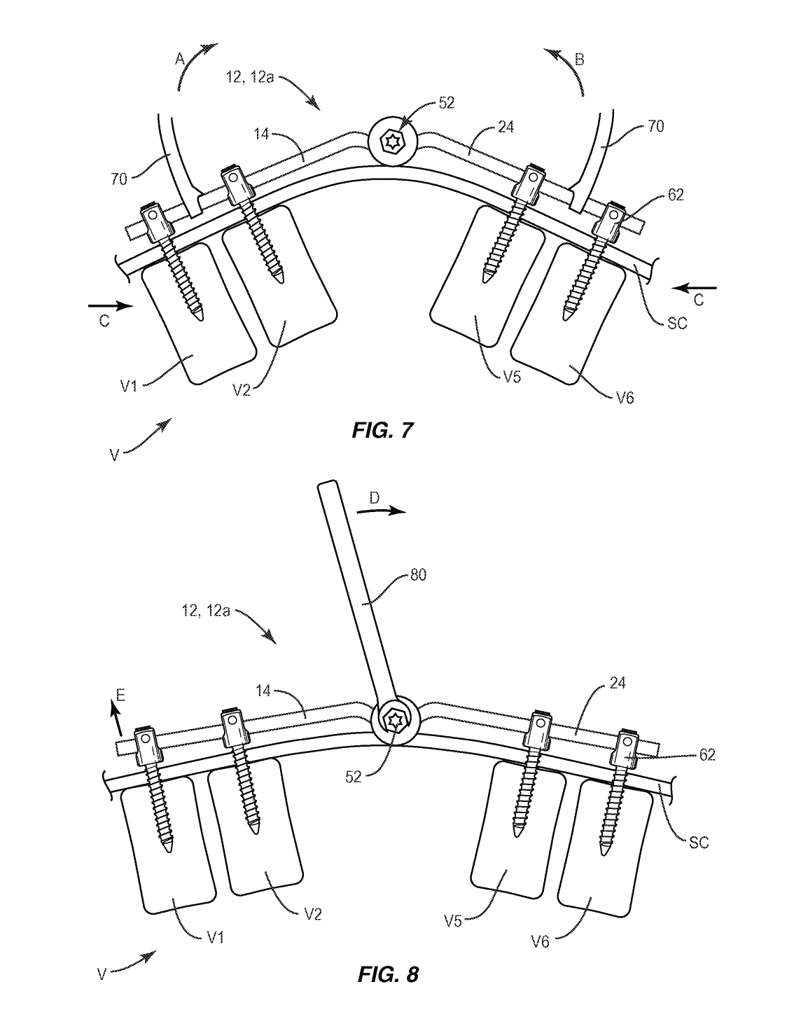

In one embodiment, as shown in FIG. 7, spinal constructs 12, 12a are disposed in a non-locking orientation with screw 52. A rod holder 70 is attached to rod 14 and a rod holder 72 is attached to rod 24 for gradual correction of vertebrae V with rotation of rods 14, 24 to one or a plurality of selected orientations to reduce kyphosis between rods 14, 24. Rod holder 70 is manipulated to rotate rod 14, in the direction shown by arrow A, and rod holder 72 is manipulated to rotate rod 24, in the direction shown by arrow B, to achieve angular correction as rod 24 rotates relative to rod 14. Spinal cord SC is shortened, in the direction shown by arrows C. Spinal constructs 12, 12a are disposable in a locking orientation with screw 52 to fix rods 14, 24 in a selected orientation. Spinal constructs 12, 12a can be locked and unlocked for incremental rotation and gradual correction of vertebrae V, as described herein.

In one embodiment, as shown in FIG. 8, spinal constructs 12, 12a are disposed in a locking orientation with screw 52. A tool 80 is attached to screw 52 for progressive correction of vertebrae V with rotation of rods 14, 24 to one or a plurality of selected orientations to reduce kyphosis between rods 14, 24. Tool 80 is manipulated, in the direction shown by arrow D to rotate rod 14, in the direction shown by arrow E, to achieve angular correction as rod 14 rotates relative to rod 24.

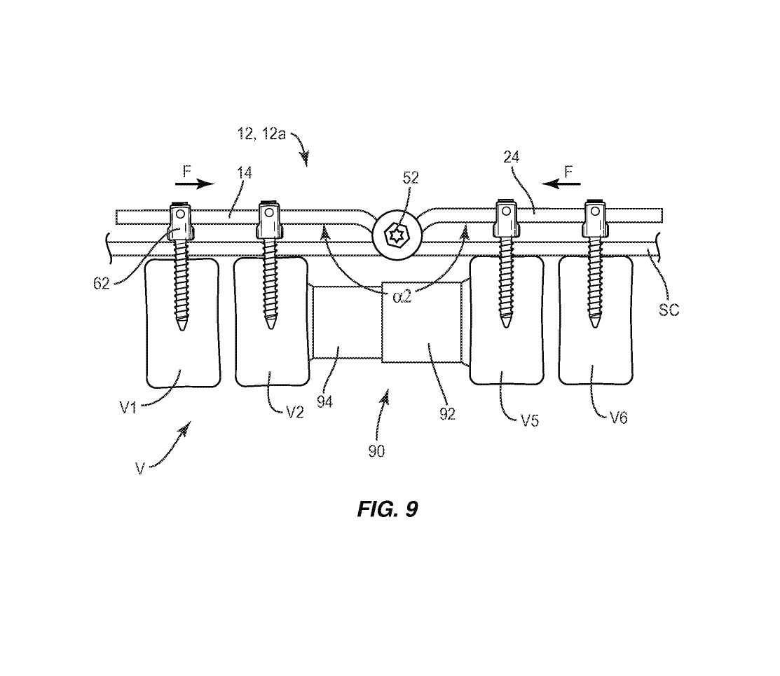

In one embodiment, as shown in FIG. 9, spinal constructs 12, 12a are disposed in a locking orientation with screw 52 to fix rods 14, 24 in a selected orientation at angle α2. Spinal implant system 10 includes an implant, such as, for example, a cage 90 having an outer body 92 and an inner body 94. Cage 90 is selectively movable between a collapsed and/or nested configuration and an expanded configuration to engage vertebrae V2, V5 adjacent vertebral soft tissue and bone surfaces to restore height and provide support in place of removed vertebrae and/or intervertebral tissue. Rods 14, 24 are compressible, in the direction shown by arrows F. In one embodiment, as shown in FIG. 10, spinal constructs 12, 12a include visual indicia, such as, for example etching 98, to display distance, for example in millimeters, for identifying shortening and/or lengthening of vertebrae V during correction.

In some embodiments, rods 14, 24 can be secured at an appropriate angle to follow the curvature of a spine or to hold a desired position of a spine. Spinal correction system 10 may be configured for disposal along a plurality of vertebral levels.

In some embodiments, the components of spinal correction system 10 may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. In some embodiments, the components of spinal correction system 10 and method of use may be used to prevent or minimize curve progression in individuals of various ages.

In one embodiment, surgical correction system 10 can include one or a plurality of bone fasteners such as those described herein and/or fixation elements, which may be engaged with tissue in various orientations, such as, for example, series, parallel, offset, staggered and/or alternate vertebral levels. In some embodiments, the bone fasteners and/or fixation elements may include one or a plurality of multi-axial screws, sagittal angulation screws, pedicle screws, mono-axial screws, uni-planar screws, fixed screws, tissue penetrating screws, conventional screws, expanding screws, wedges, anchors, buttons, clips, snaps, friction fittings, compressive fittings, expanding rivets, staples, nails, adhesives, posts, fixation plates and/or posts. In some embodiments, surgical correction system 10 may comprise various instruments, such as, for example, inserters, extenders, reducers, spreaders, distractors, blades, retractors, clamps, forceps, elevators and drills, which may be alternately sized and dimensioned, and arranged as a kit.

In some embodiments, surgical correction system 10 includes an agent, which may be disposed, coated, packed or layered within, on or about the components and/or surfaces of surgical correction system 10. In some embodiments, the agent may include bone growth promoting material, such as, for example, bone graft to enhance fixation of the cage and/or fixation elements with vertebrae V. The components of surgical correction system 10 can be made of radiolucent materials such as polymers. Radiomarkers may be included for identification under x-ray, fluoroscopy, CT or other imaging techniques. In some embodiments, the agent may include one or a plurality of therapeutic agents and/or pharmacological agents for release, including sustained release, to treat, for example, pain, inflammation and degeneration. Upon completion of the procedure, the surgical instruments, assemblies and non-implant components of surgical correction system 10 are removed from the surgical site and the incision is closed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal construct comprising:
a first member defining a longitudinal axis and extending between a first end and a second end being offset relative to the longitudinal axis, the first member being configured for attachment to at least a first vertebra of a body; and
a second member being configured for attachment to at least a second vertebra of the body spaced from the first vertebra and defining a longitudinal axis, the second member extending between a first end and a second end being offset relative to the longitudinal axis of the second member, the second ends being connected to define an axis of rotation such that the members are relatively rotatable about the second ends and the axis of rotation is offset relative to the longitudinal axes.

2. A spinal construct as recited in claim 1, wherein the first member is attached to a posterior portion of the first vertebra and the second member is attached to a posterior portion of the second vertebra such that the axis of rotation is offset anterior to the longitudinal axes.

3. A spinal construct as recited in claim 1, wherein the second ends are offset relative to the longitudinal axes in a sagittal plane of the body.

4. A spinal construct as recited in claim 1, wherein the second end of the first member is offset relative to its longitudinal axis in a coronal plane of the body.

5. A spinal construct as recited in claim 1, wherein the first member includes a rod portion extending between its ends, the rod portion including an arcuate portion configured to offset the second end of the first member relative to its longitudinal axis.

6. A spinal construct as recited in claim 1, wherein the first member includes a rod portion extending between its ends, the rod portion including an arcuate portion configured to offset the second end of the first member relative to its longitudinal axis and the second member includes a rod portion extending between its ends, the rod portion of the second member including an arcuate portion configured to offset the second end of the second member relative to its longitudinal axis.

7. A spinal construct as recited in claim 1, further comprising a lock connected to the second ends to dispose the members in a selected orientation.

8. A spinal construct as recited in claim 1, further comprising a lock connected to the second ends to dispose the members in a selected orientation, the lock including a resiliently biased member configured to bias the lock to a non-locking orientation.

9. A spinal construct as recited in claim 1, wherein the second end of the first member includes a mating surface configured to interface with a mating surface of the second end of the second member to dispose the members in a selected orientation.

10. A spinal construct as recited in claim 1, wherein the second ends each include a transverse surface having a plurality of teeth disposed radially thereabout, the teeth being configured to interface for fixation of the members in a selected orientation.

11. A spinal construct as recited in claim 10, further comprising a ratchet including the teeth such that the members are incrementally rotatable to one or a plurality of selected orientations.

12. A spinal construct as recited in claim 10, further comprising a ratchet including the teeth such that the members are rotatable in 5 degree increments to one or a plurality of selected orientations.

13. A spinal construct as recited in claim 1, wherein the second ends each include a transverse surface having a radial spline configuration, the transverse surfaces being configured to interface for fixation of the members in a selected orientation.

14. A spinal construct as recited in claim 1, wherein the members are relatively rotatable in a sagittal plane of the body between a first configuration such that the members are disposed at a first relative angular orientation and a second configuration such that the members are disposed at a second relative angular orientation.

15. A spinal construct as recited in claim 1, wherein in the second configuration, the members are disposed in substantially coaxial alignment.

16. A spinal construct as recited in claim 1, wherein each second end includes a disc shaped plate, the plates being connected in a hinge configuration to facilitate relative rotation of the members.

17. A spinal construct comprising:
a first rod defining a longitudinal axis and extending between a first end and a second end, the second end including a plate being offset relative to the longitudinal axis, the first rod being configured for attachment to a posterior portion of first vertebrae of a body; and
a second rod being configured for attachment to a posterior portion of second vertebrae of the body spaced from the first vertebrae and defining a longitudinal axis, the second rod extending between a first end and a second end, the second end including a plate being offset relative to the longitudinal axis of the second rod, the plates being connected to define an axis of rotation such that the rods are relatively rotatable about the plates such that the axis of rotation is offset anterior to the longitudinal axes;
wherein the rods are rotatable in a sagittal plane of the body between a first configuration such that the rods are disposed at a first relative angular orientation and a second configuration such that the rods are disposed at a second relative angular orientation.

18. A spinal construct as recited in claim 17, further comprising a lock connected to the plates to dispose the rods in a selected orientation.

19. A spinal construct as recited in claim 17, wherein the plate of the first rod is offset relative to its longitudinal axis in a coronal plane of the body.

20. A spinal construct comprising:
a lateral rod defining a longitudinal axis and extending between a first end and a second end, the second end including a plate being offset relative to the longitudinal axis in a sagittal plane of a body and a coronal plane of the body, the plate including a plurality of teeth disposed radially thereabout, the lateral rod being configured for attachment to a posterior portion of first vertebrae of a body;
a medial rod being configured for attachment to a posterior portion of second vertebrae of the body spaced from the first vertebrae and defining a longitudinal axis, the second rod extending between a first end and a second end, the second end including a plate being offset relative to the longitudinal axis of the second rod, the plate of the second rod including a plurality of teeth disposed radially thereabout,
the plates being connected to define an axis of rotation such that the rods are relatively rotatable about the plates such that the axis of rotation is offset anterior to the longitudinal axes; and
a lock connected to the plates and being configured to interface the teeth such that the rods are incrementally rotatable and fixable in one or a plurality of selected orientations.
